(19) United States
(12) Patent Application Publication
(10) Pub. No.: US 2006/0191540 A1
(43) Pub. Date: Aug. 31, 2006

(54) Disposable medical or surgical procedure drape and attached instruments

(76) Inventors: Lonnie Jay Lamprich, Oklahoma City, OK (US); Bradley Keith Lamprich, Oklahoma City, OK (US)

Correspondence Address: Clifford C. Dougherty, III;McAfee & Taft, Tenth Floor, Two Leadership Square, 211 North Robinson, Oklahoma City, OK, 73102, US

(21) Appl. No.: 11/393,123

(22) Filed: Mar. 29, 2006

Related U.S. Application Data
(63) Continuation-in-part of application No. 10/955,766, filed on Sep. 30, 2004.

(51) Int. Cl. A61B 19/00; A61B 19/08; A61F 5/37

(52) U.S. Cl. 128/849; 128/853

(57) ABSTRACT

A disposable sterile medical or surgical procedure drape with pre-attached disposable sterile instruments for use in a selected medical or surgical procedure is provided. The disposable sterile medical or surgical procedure drape basically comprises a drape having a size for covering an operating table and a patient thereon and having a fenestration formed therein, a patient heat distributing pad pre-attached to the bottom side of the drape, an absorbent cloth surrounding the fenestration pre-attached to the bottom side of the drape, a cover for the fenestration in the drape pre-attached to the top side of the drape, and the surgical instruments and connecting tubes or wires required to accomplish the medical or surgical procedure pre-attached to the drape.

This application is a continuation-in-part of Ser. No. 10/955,766, filed Sep. 30, 2004.

### BACKGROUND OF THE INVENTION

1. Field of the Invention

The present invention provides a disposable sterile drape with attached disposable sterile instruments for use in selected surgical, diagnostic, interventional and other medical procedures.

2. Description of the Prior Art

Medical or surgical procedure drapes, often called surgical drapes, are used during surgical and other medical procedures to create and maintain a sterile environment around the site of the procedure. The drape creates and maintains a barrier that minimizes the passage of microorganisms between non-sterile and sterile areas. To be effective, the drape material must be resistant to blood, aqueous fluid and abrasion as well as being lint free. As a result, the drape prevents blood and other bodily fluids from contaminating the sterile field; for example, the incision and the patient's skin surrounding the incision.

Medical or surgical procedure drapes were originally formed of cotton or linen and were sterilized after each use. More recently, disposable drapes have been utilized formed of paper or fabric. The drapes commonly have an opening formed therein known in the medical field as a fenestration through which the surgical or other medical procedure is performed.

For example, in a surgical procedure, after a patient has been anesthetized in an operating room, the surgical team individually places the various components of the sterile drape and the instruments required on the patient. That is, a sterile heat distributing pad is placed on the patient to keep the patient warm followed by sterile absorbent towels which are placed on the patient around the surgical field. Thereafter, the sterile drape is placed on the patient so that the incision opening or fenestration is positioned over the surgical field. A cover for the fenestration which is impregnated with iodine to kill bacteria is attached to the drape adjacent to the fenestration. The cover, which is transparent and includes an adhesive on one side, is positioned over the surgical field whereby it adheres to the drape and to the patient's skin within the surgical field. Finally, the various sterile surgical instruments and connecting tubes or wires required to accomplish the surgical procedure are placed on the sterile drape one at a time. This procedure of preparing the patient for surgery in the operating room after the patient has been anesthetized involves a lengthy period of time. Typically, the patient preparation time can take as long as forty-five minutes during which the patient is anesthetized.

Thus, there is a need for a patient preparation procedure utilizing sterile drape components and sterile instruments that does not require the patient and surgical team to be in the operating room for a long period of time before the medical or surgical procedure can be performed. In addition, there is a need for a sterile drape having the various components pre-attached to the drape whereby there is little or no handling of the components by technicians or others not familiar with the components and as a result, the sterility of the drape and components is insured. Finally, there is a need for a sterile drape whereby the tubes and wires connected to the various sterile medical or surgical procedure instruments are attached to the drape between sterile layers of the drape whereby the tubes and wires are not contaminated from below or above the drape.

### SUMMARY OF THE INVENTION

The present invention provides improved disposable sterile medical or surgical procedure drapes having disposable sterile instruments attached thereto for use in selected surgical and other medical procedures which meet the needs described above and overcome the deficiencies of the prior art.

A disposable sterile medical or surgical procedure drape of this invention having disposable sterile instruments attached thereto for use in a selected medical or surgical procedure is comprised of the following components. A drape is provided having a size for covering an operating table and a patient thereon and having an enlargeable fenestration formed therein. An absorbent cloth surrounding the fenestration is pre-attached to the bottom side of the drape and a patient heat distributing pad is also pre-attached to the bottom side of the drape whereby it is positioned adjacent to the patient. A cover for the fenestration in the drape is pre-attached to the top side of the drape adjacent to the fenestration. One or more instruments and one or more connecting tubes or wires for use in the medical or surgical procedure are pre-attached to the top side of the drape.

The disposable sterile drape having disposable sterile instruments for use in a selected medical or surgical procedure attached thereto is provided in a package which maintains the sterility of the drape and its attachments. The package is opened in the operating room and the disposable sterile medical or surgical procedure drape including attached disposable sterile instruments for a particular medical or surgical procedure is quickly and easily placed on the patient and the patient is ready for the medical or surgical procedure in a very short period of time thereafter.

A disposable sterile drape with attached disposable sterile instruments for use in a lumbar surgical procedure comprises the following components. A drape is provided having a size for covering an operating table and a patient thereon and having an enlargeable fenestration formed therein. A patient heat distributing pad is pre-attached to the bottom side of the drape and an absorbent cloth surrounding the enlargeable fenestration is pre-attached to the bottom side of the drape. A sterile cover for the enlargeable fenestration in the drape is pre-attached to the top side of the drape adjacent to the fenestration. Finally, the surgical instruments and connecting tubes and wires required to perform the lumbar surgical procedure are attached to the top side of the drape.

The objects, features and advantages of the present invention will be readily apparent to those skilled in the art upon a reading of the description of preferred embodiments which follows when taken in conjunction with the accompanying drawings.

### BRIEF DESCRIPTION OF THE DRAWINGS

FIG. 1 is a view of a patient on an operating table with the medical or surgical procedure drape of the present invention placed thereon.

FIG. 2 is a view of a square enlargeable fenestration formed in the medical or surgical procedure drape of FIG. 1.

FIG. 3 is a view of a rectangular enlargeable fenestration formed in the medical or surgical procedure drape of FIG. 1.

FIG. 4 is a top view of the medical or surgical procedure drape of this invention having the various components required for a lumbar surgical procedure pre-attached thereto.

### DESCRIPTION OF PREFERRED EMBODIMENTS

A disposable sterile drape having pre-attached disposable sterile instruments of this invention for use in a selected medical or surgical procedure is illustrated in the drawings. As used herein and in the appended claims, a “fenestration” means an incision opening or other opening in the inventive drape through which the medical or surgical procedure is performed. An “operating table” means a table or other structure on which the patient is disposed for the medical or surgical procedure. An “operating room” means the room in which the medical or surgical procedure is carried out. Examples of types of procedures in which the inventive drape can be used include surgical procedures, diagnostic procedures and interventional medical procedures. Specific examples of surgical procedures in which the inventive drape can be used include lumbar surgeries, laparotomy surgeries, thoracotomy surgeries, median sternotomy surgeries, extremity surgeries, urologic surgeries and gynecologic surgeries. Specific examples of other types of medical procedures in which the inventive drape can be used include diagnostic biopsies (e.g., image guided biopsies), endoscopies, and cardiac and other catheterization procedures.

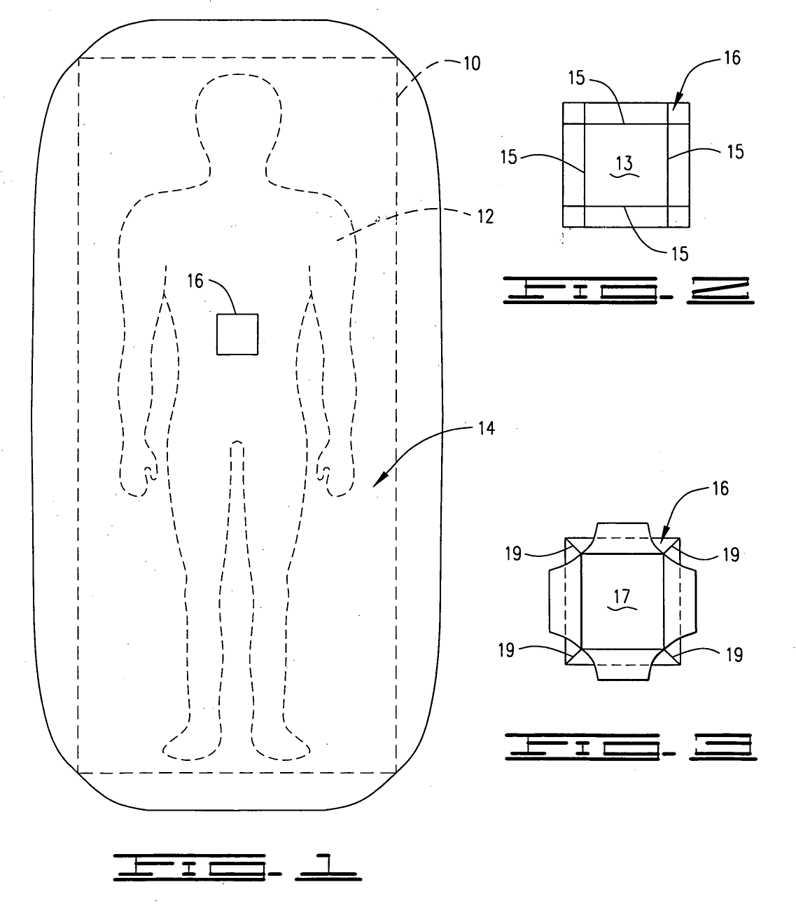

FIG. 1 is a top view of an operating table 10 having a patient 12 lying face down on the table 10. The table 10 and patient 12 are completely covered with a disposable sterile medical or surgical procedure drape 14 of this invention having a square enlargeable fenestration 16 therein. The enlargeable fenestration 16 in FIG. 1 is an incision opening positioned for lumbar surgery from the patient's back.

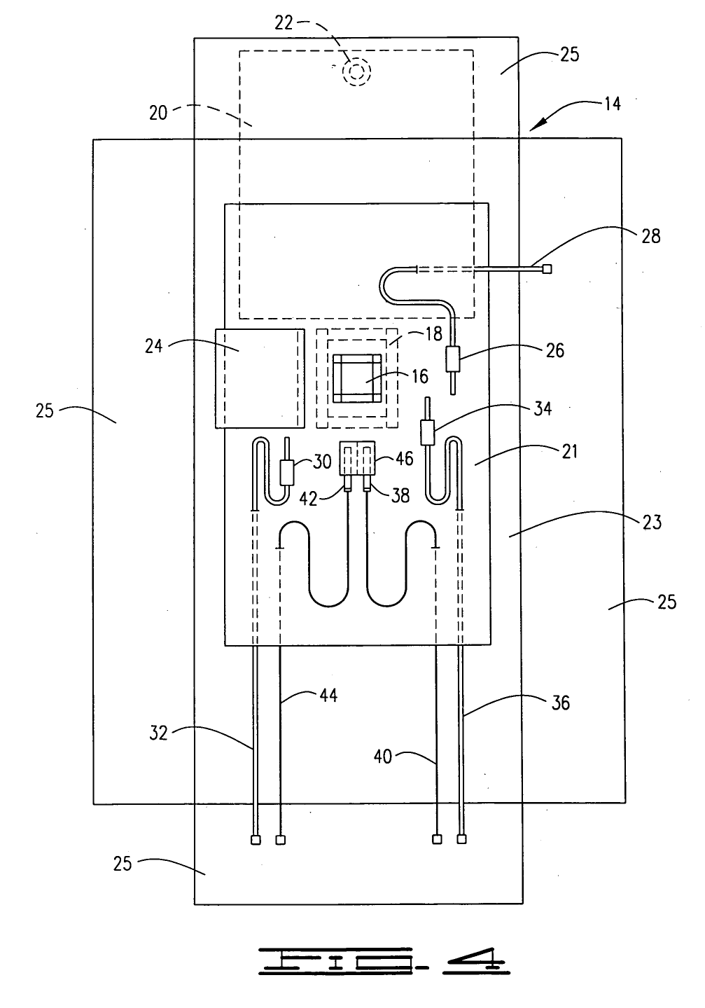

The disposable sterile drape 14 is preferably formed of two attached layers of paper, a top layer 21 which is plastic treated, and a bottom layer 23 having a skirt formed of four rectangular paper parts 25 attached thereto. The fenestration 16 is preferably square or rectangular. As illustrated in FIG. 2, the fenestration 16 can be a square opening 13 with slits 15 forming top, bottom and side flaps that can be folded back to form enlarged square or rectangular openings. As illustrated in FIG. 3, the fenestration 16 can be a rectangular opening 17 having 45° slits 19 extending from the corners thereof whereby the opening 17 can be enlarged by folding back the sides of the opening. Other fenestration shapes can also be utilized.

Referring now to FIG. 4, the disposable sterile drape 14 with pre-attached disposable sterile instruments and other components is illustrated. As described above, the disposable sterile drape 14 includes an enlargeable fenestration 16 formed therein.

A patient heat distributing pad 20 having heated air openings or baffles therein and a tube connection 22 for providing heated air thereto is attached to the bottom side of the drape 14. When the medical or surgical procedure drape 14 is utilized, the patient heat distributing pad 20 is connected to a source of heated air by way of a tube (not shown) connected to the tube connection 22, and heated air is circulated through the pad. As will be understood by those skilled in the art, the heated air circulated through the heat distributing pad 20 keeps the patient warm during the medical procedure. The heat distributing pad 20 includes an adhesive on the side adjacent to the patient whereby it adheres to the patient during the medical procedure. Examples of heat distributing pads that can be used as the patient heat distributing pad 20 are sold by Arizant Healthcare, Inc in association with the trademark “BAIR HUGGER®.”

An absorbent cloth 18 preferably formed of several layers of toweling is also pre-attached to the bottom side of the drape 14 positioned around the enlargeable fenestration 16. The absorbent cloth 18 provides a barrier to absorb blood and other fluids during surgery.

A cover 24 for covering the fenestration 16 is pre-attached to the top side of the drape 14 along one side thereof adjacent to the fenestration. The cover 24 is preferably impregnated with iodine or another anti-bacterial composition to kill bacteria. An example of an anti-bacterial composition that can be used is “BETADINE®” Microbicides (povidone-iodine). The cover 24 is transparent and includes an adhesive on the top side so that it covers the fenestration 16 and sticks to the portion of the drape 14 containing the fenestration and to the patient's exposed skin to hold the drape securely to the patient.

Depending upon the particular medical or surgical procedure to be carried out, the disposable sterile instruments and connecting tubes or wires for use in the procedure, or at least some of the instruments and tubes or wires, are pre-attached to the drape 14. As mentioned above, the drape 14 is sterile as are all the parts pre-attached to the drape including the instruments required to carry out the medical procedure.

A specific procedure which utilizes the disposable sterile drape 14 illustrated in the drawing is a posterior lumbar surgery procedure. The sterile drape and instruments for the posterior lumbar surgery are illustrated in FIG. 4. More specifically, the surgical instruments and connecting tubes and wires required to perform the lumbar surgical procedure are pre-attached to the drape. That is, a portion of each tube and wire is attached between the top layer 21 and the bottom layer 23 of the drape so that the instruments are prevented from falling to the floor when not being used. The instrument 26 attached to the drape is a cell-saver suction instrument with a connecting tube 28. A first suction instrument 30 with a connecting tube 32 and a second suction instrument 34 with a connecting tube 36 are also attached to the drape 14. A bipolar cautery instrument 38 (less tip) having a connecting wire 40 and a monopolar cautery instrument 42 having a wire 44 are also attached to the drape 14. An instrument pocket 46 for the two cautery instruments is also attached to the top of the drape 14.

As will be understood by those skilled in the art, the tubes 28, 32 and 36 of the suction instruments are manually connected to additional tubes that connect to one or more suction machines. The wires 40 and 44 attached to the cautery instruments 38 and 42 are manually connected to additional wires that provide electricity thereto. When the surgical procedure has been accomplished, the tubes and wires are disconnected and the surgical drape 14 is removed from the patient. The surgical drape as well as the attached instruments, tubes and wires are then disposed.

As will now be understood, disposable sterile medical or surgical procedure drapes of this invention with pre-attached instruments for performing various medical procedures are provided in sealed packages. When a sterile drape is needed for a particular medical or surgical procedure, the appropriate package is opened in the operating room and the sterile drape with pre-attached instruments is removed therefrom and quickly utilized. After being utilized, the package as well as the sterile drape and its attachments are disposed. A disposable sterile drape with attached disposable sterile instruments for use in a selected medical or surgical procedure of this invention comprises: a drape having a size for covering an operating table and a patient thereon and having a fenestration formed therein; a patient heat distributing pad pre-attached to the bottom side of the drape; an absorbent cloth surrounding the fenestration pre-attached to the bottom side of the drape; a cover for the fenestration in the drape pre-attached to the top side of the drape adjacent to the fenestration; and one or more medical or surgical procedure instruments (for example, surgical instruments) and one or more connecting tubes or wires for use in the medical or surgical procedure pre-attached to the top side of the drape. In one embodiment, substantially all of the medical or surgical procedure instruments and connecting tubes or wires required to accomplish the medical or surgical procedure are pre-attached to the drape.

A disposable sterile drape with attached disposable sterile instruments for use in a lumbar surgical procedure of this invention comprises: a drape having a size for covering an operating table and a patient thereon and having an enlargeable fenestration formed therein; a patient heat distributing pad attached to the bottom side of the drape; an absorbent cloth surrounding the enlargeable fenestration pre-attached to the bottom side of the drape; a cover for the enlargeable fenestration in the drape pre-attached to the top side of the drape adjacent to the fenestration; and the surgical instruments and connecting tubes and wires required to perform the lumbar surgical procedure pre-attached to the top side of the drape.

As will be understood by those skilled in the art, numerous changes may be made to the disposable sterile medical or surgical procedure drape with pre-attached instruments of the present invention without departing from the spirit and scope of the invention as defined by the appended claims. For example, one or more fenestrations of various shapes can be included in the disposable sterile medical or surgical procedure drape of this invention and a variety of medical or surgical procedures can be conducted using the drape.

What is claimed is:

1. A disposable sterile medical or surgical procedure drape with pre-attached disposable sterile instruments for use in a selected medical or surgical procedure comprising:
a drape having a size for covering an operating table and a patient thereon and having a fenestration formed therein;
a patient heat distributing pad pre-attached to the bottom side of the drape;
an absorbent cloth surrounding the fenestration pre-attached to the bottom side of the drape;
a cover for the fenestration in the drape pre-attached to the top side of the drape adjacent to the fenestration; and
one or more instruments and one or more connecting tubes or wires for use in the medical or surgical procedure pre-attached to the drape.

2. The disposable sterile medical or surgical procedure drape of claim 1 wherein the drape is formed of two layers of paper, the top layer being plastic treated.

3. The disposable sterile medical or surgical procedure drape of claim 1 wherein the drape includes a skirt formed of four rectangular paper parts.

4. The disposable sterile medical or surgical procedure drape of claim 1 wherein the fenestration is a square or rectangular opening.

5. The disposable sterile medical or surgical procedure drape of claim 1 wherein the fenestration is square and has top, bottom and side flaps that can be folded back to form an enlarged square or rectangular opening.

6. The disposable sterile medical or surgical procedure drape of claim 3 wherein the fenestration is rectangular and includes 45° slits extending from the corners thereof whereby the opening can be enlarged by folding back the sides of the opening.

7. The disposable sterile medical or surgical procedure drape of claim 1 wherein the heat distributing pad comprises heated air openings therein and a tube connection for providing heated air thereto.

8. The disposable sterile medical or surgical procedure drape of claim 1 wherein the heat distributing pad includes an adhesive on the side adjacent to the patient whereby it adheres to the patient.

9. The disposable sterile medical or surgical procedure drape of claim 1 wherein the absorbent cloth surrounding the fenestration is formed of toweling.

10. The disposable sterile medical or surgical procedure drape of claim 1 wherein the cover for the fenestration is impregnated with an anti-bacterial composition to kill bacteria, is transparent and includes an adhesive on the side adjacent to the patient whereby it adheres to the fenestration and to the patient's exposed skin.

11. The disposable sterile medical or surgical procedure drape of claim 1 which further comprises a package for containing the disposable sterile medical or surgical procedure drape and the attachments thereto and for maintaining the sterility thereof.

12. The disposable sterile medical or surgical procedure drape of claim 1 wherein the selected medical or surgical procedure is a lumbar surgery, laparotomy surgery, thoracotomy surgery, median sternotomy surgery, extremity surgery, urologic surgery, gynecologic surgery or other surgery.

13. A disposable sterile surgical drape with pre-attached disposable sterile instruments for use in a lumbar surgical procedure comprising:
a drape having a size for covering an operating table and a patient thereon and having an enlargeable fenestration formed therein;
a patient heat distributing pad pre-attached to the bottom side of the drape;
an absorbent cloth surrounding the enlargeable fenestration pre-attached to the bottom side of the drape;
a cover for the enlargeable fenestration in the drape pre-attached to the top side of the drape adjacent to the fenestration; and
the surgical instruments and connecting tubes and wires required to perform the lumbar surgical procedure pre-attached to the drape.

14. The disposable sterile surgical drape of claim 13 wherein a cell-saver suction instrument with connecting tube is pre-attached to the drape.

15. The disposable sterile surgical drape of claim 13 wherein a first suction instrument with connecting tube is also pre-attached to the drape.

16. The disposable sterile surgical drape of claim 13 wherein a second suction instrument with connecting tube is also pre-attached to the drape.

17. The disposable sterile surgical drape of claim 13 wherein a bipolar cautery instrument with connecting wire is also pre-attached to the drape.

18. The disposable sterile surgical drape of claim 13 wherein a monopolar cautery instrument with connecting wire is also pre-attached to the drape.

19. The disposable sterile surgical drape of claim 13 wherein the surgical drape is formed of two layers of paper, the top layer being plastic treated.

20. The disposable sterile surgical drape of claim 13 wherein the drape includes a skirt formed of four rectangular paper parts.

21. The disposable sterile surgical drape of claim 13 wherein the fenestration is a square or rectangular opening.

22. The disposable sterile surgical drape of claim 13 wherein the fenestration is square and has top, bottom and side flaps that can be folded back to form an enlarged square or rectangular opening.

23. The disposable sterile surgical drape of claim 13 wherein the fenestration is rectangular and includes 45° slits extending from the corners thereof whereby the opening can be enlarged by folding back the sides of the opening.

24. The disposable sterile surgical drape of claim 13 wherein the heat distributing pad comprises heated air openings therein and a tube connection for providing heated air thereto.

25. The disposable sterile surgical drape of claim 13 wherein the heat distributing pad includes an adhesive on the side adjacent to the patient whereby it adheres to the patient.

26. The disposable sterile surgical drape of claim 13 wherein the absorbent cloth surrounding the enlargeable incision opening is formed of toweling.

27. The disposable sterile surgical drape of claim 13 wherein the cover for the incision opening is impregnated with an anti-bacterial composition to kill bacteria, is transparent and includes an adhesive on the side adjacent to the patient whereby it adheres to the enlargeable incision opening and to the patient's exposed skin.

28. The disposable sterile surgical drape of claim 13 which further comprises a package for containing the disposable sterile surgical drape and the pre-attachments thereto and for maintaining the sterility thereof.
